(19) United States
(12) Patent Application Publication
(10) Pub. No.: US 2026/0157632 A1
(43) Pub. Date: Jun. 11, 2026

(54) Radiofrequency Shielding Facility for a Dedicated Magnetic Resonance Device

(71) Applicant: Siemens Healthineers AG, Forchheim (DE)

(72) Inventors: Lars Lauer, Neunkirchen (DE); Gunnar Krüger, Binningen (CH); Andreas Greiser, Erlangen (DE); Miriam Keil, Erlangen (DE); Annemarie Hausotte, Erlangen (DE); Rainer Schneider, Höchstadt (DE)

(73) Assignee: Siemens Healthineers AG, Forchheim (DE)

(21) Appl. No.: 19/408,458

(22) Filed: Dec. 4, 2025

(30) Foreign Application Priority Data
Dec. 5, 2024 (DE) 10 2024 211 635.1

(51) Int. Cl. A61B 5/00

(52) U.S. Cl. CPC A61B 5/0046; A61B 5/004

(57) ABSTRACT

A radiofrequency (RF) shielding facility is provided for a dedicated magnetic resonance device, for example a dedicated MRT head scanner. The RF shielding facility may for instance comprise modular radiofrequency shielding elements configured to be connected to one another and in the interconnected state to form a space for arranging the dedicated magnetic resonance device, and/or at least one RF shield configured to be arranged on one side on a patient and/or on a movable patient seat, and on another side on the dedicated magnetic resonance device, and/or at least one RF antenna configured to at least partially cancel a RF signal of the dedicated magnetic resonance device via destructive interference.

### CROSS-REFERENCE TO RELATED APPLICATIONS

The present application claims priority to and the benefit of Germany patent application no. DE 10 2024 211 635.1, filed on Dec. 5, 2024, the contents of which are incorporated herein by reference in their entirety.

### TECHNICAL FIELD

The present disclosure relates to a radiofrequency (RF) shielding facility for a dedicated magnetic resonance device, e.g. for a dedicated MRT head scanner, and to a magnetic resonance arrangement comprising such a RF shielding facility.

### BACKGROUND

Magnetic resonance tomography systems or magnetic resonance scanners are imaging devices which, to image an examination subject, align nuclear spins of the examination subject by means of a strong external magnetic field and excite said nuclear spins by means of an alternating magnetic field into precession around said alignment. The precession or, as the case may be, the return of the spins from said excited state into a state having lower energy, in turn generates as response an alternating magnetic field which is received via antennas.

A spatial encoding scheme is superimposed on the signals with the aid of magnetic gradient fields, which spatial encoding scheme subsequently enables the received signal to be assigned to a volume element. The received signal is then evaluated, and a three-dimensional imaging representation of the examination subject is provided.

To deploy dedicated magnetic resonance devices, such as dedicated head scanners or dedicated dental scanners in smaller medical institutions and practices, the smallest possible space requirement is desired. In this case it is important to keep the necessary installation area as small as possible. One of the main reasons for the greater space requirement of magnetic resonance arrangements is the need for RF shielding of the magnetic resonance device.

Basically, the entire space in which a magnetic resonance device is installed is designed as an RF cage and is provided with RF attenuation for the respective frequencies of the respective transmit/receive bandwidth of the magnetic resonance device. Typically, copper plates or copper meshes are provided in this arrangement on all of the walls, the floor, and the ceiling, as well as a shielded door and windows with shielding glass.

### SUMMARY

In this connection it has become clear that there is a need to provide a simpler means of RF shielding for a dedicated magnetic resonance device, e.g. for a dedicated MRT head scanner.

It is therefore an object of the present disclosure to provide a solution for enabling a RF shield for a dedicated magnetic resonance device to be provided more easily, e.g. for a dedicated MRT head scanner.

These and other objects cited in the course of the reading of the following description, or which may be identified by the person skilled in the art are achieved by means of the subject matter of as described herein, including the claims. The claims may describe the central idea of the present disclosure in a particularly advantageous manner.

A first aspect of the present disclosure relates to a RF shielding facility for a dedicated magnetic resonance device, e.g. for a dedicated MRT head scanner, comprising:
modular RF shielding elements which are configured to be connected to one another and in the interconnected state to form a room for arranging the dedicated magnetic resonance device, and/or
at least one RF shield which is configured to be arranged on one side on a patient and/or on a movable patient seat, and on another side on the dedicated magnetic resonance device, and/or
at least one RF antenna which is configured to extinguish (partially or fully cancel or otherwise attenuate) a RF signal of the dedicated magnetic resonance device by means of destructive interference.

In other words, the present disclosure relating to the RF shielding of a dedicated magnetic resonance device proposes the use of at least one of the three cited shielding techniques, wherein the shielding techniques may be used independently or be combined with one another.

The RF shielding elements may e.g. be designed as self-supporting planar components which comprise connecting means to enable said components to be connected to one another in the simplest possible manner. In this arrangement, the RF shielding elements can be designed as standardized components, in which case different functions can be provided by means of different RF shielding elements. For example, standardized door elements, window elements, wall, ceiling, and floor elements and the like can be provided and adapted to suit different installation space requirements.

A kind of room-within-a-room concept can easily be provided by means of the RF shielding elements. In a simple embodiment, the RF shielding elements effectively enable a shielding cabin or a shielding cage to be constructed in which the dedicated magnetic resonance device can be arranged completely or at least in part. The RF shielding elements can in this case comprise for example a metallic web, for example a copper mesh and/or metal plates, such as, for example, copper plates and other RF attenuating elements.

In an exemplary embodiment of a RF shielding facility, the latter comprises modular RF shielding elements that are configured to be connected to one another and, in the interconnected state, to form a room for arranging the dedicated magnetic resonance device, and at least one RF antenna which is configured to extinguish a RF signal of the dedicated magnetic resonance device by means of destructive interference.

In such an embodiment, the requirements in respect of the shielding by means of the RF shielding elements or, with respect to the room (e.g. any suitable space) constructed by these, can be reduced such that the RF shielding elements can be provided more easily, more simply, and also more cheaply so that overall the construction of the shielding cabin can be simplified and provided more cost-effectively. The higher RF leakage potentially occurring due to the lower requirements in terms of the shielding of the shielding cabin can be determined comparatively precisely as a result of a standardized construction of the shielding cabin, such that said leakage can be neutralized by means of destructive interference through the use of RF antennas. In this case the RF antennas can be arranged outside of the shielding cabin, these may e.g. be integrated into the RF shielding elements or be secured thereto.

In an exemplary embodiment of a RF shielding facility, the latter comprises at least one RF shield configured to be arranged on one side on a patient and/or on a patient seat, and on another side to be arranged on the dedicated magnetic resonance device, and at least one RF antenna which is configured to extinguish a RF signal of the dedicated magnetic resonance device by means of destructive interference.

Such an embodiment comprising a RF shield may be advantageously employed when the dedicated magnetic resonance device comprises a one-sided scanning bore opening, which is configured to accommodate at least a head of a patient. In this embodiment, the side of the magnetic resonance device opposite the scanning bore opening may be permanently closed and may e.g. already be adequately shielded. In such an embodiment, the RF shield serves to shield the part of the patient arranged outside the scanning bore opening from the part of the patient arranged inside the scanning bore opening. The RF leakage potentially occurring in this case can in turn be neutralized by means of destructive interference through the use of RF antennas.

The RF shield can in this case be designed for example as a stopper-like ring structure closing (e.g. physically and/or in at least a partially RF-sealing manner) the scanning bore opening and moving together with the patient bed or patient seat in the magnetic resonance device during the positioning. A RF shield, for example in the form of a cork-like ring structure, can be attached for example to a metal web, advantageously covered with soft material or embedded therein, which either completely covers the body, the arms and the legs of the patient, or is wrapped around the neck of the patient during the positioning. The RF shield may e.g. be implemented as a flexible element. The RF shield and the patient may be advantageously grounded to further suppress a RF coupling.

The present disclosure further relates to a magnetic resonance arrangement comprising a dedicated magnetic resonance device and at least one RF shielding facility as described above.

In an exemplary embodiment of the magnetic resonance arrangement, the dedicated magnetic resonance device comprises a one-sided scanning bore opening which is configured to accommodate at least a head of a patient. As already stated, the side of the magnetic resonance device opposite the scanning bore opening may be e.g. permanently closed and is already adequately shielded against RF interference. In such an embodiment, it is simply necessary to shield the side of the scanning bore opening against RF interference.

In such an embodiment of the dedicated magnetic resonance device having a one-sided scanning bore opening, at least one RF shield can be provided which can be arranged on one side on a patient and/or on a patient seat and on another side on the dedicated magnetic resonance device to close (e.g. physically and/or providing at least a partial RF seal) the one-sided scanning bore opening against RF interference. By an arrangement, in this context e.g. a releasable connection may be provided by means of appropriate holding means, such as, for example, hook-and-loop fasteners, zippers, pressure connections, and the like.

In an exemplary embodiment of the magnetic resonance arrangement, the magnetic resonance arrangement further comprises a patient seat and modular RF shielding elements, which are connected to one another and form a room (e.g. a space) around the one-sided scanning bore opening of the magnetic resonance device and the patient seat. In an embodiment of the dedicated magnetic resonance device having a one-sided scanning bore opening, there may only be the need to shield the side having the scanning bore opening against RF interference. As a result, it is possible to arrange the room to be constructed by means of the modular RF shielding elements such that it does not contain the magnetic resonance device completely, but covers only the part of the magnetic resonance device having the scanning bore opening. This affords the opportunity to achieve a further reduction in the necessary installation space for a shielding cabin. In other words, this enables the shielding cabin to be designed smaller, and shielding material can be saved as a result since the magnet of the magnetic resonance device already serves as shielding on its closed side. An embodiment comprising a shielding cabin also enables a more pleasant patient experience to be provided compared to the use of a RF shield which must be arranged on one side on a patient and/or comparatively close to a patient on the patient seat.

Finally, it is also possible to adapt the shape of the shielding cabin to fit a movement space of a patient positioning apparatus. For example, it is possible to provide a shielding cabin which opens toward one side of the patient chair and is implemented as closed in the two other directions. By this means, no additional room length is required toward the foot end and the typical path for accessing the patient chair in a dental practice can be emulated as a result.

The present disclosure further relates to a use of at least one modular RF shielding element, and/or of at least one RF shield and/or of at least one RF antenna as a RF shielding facility in a magnetic resonance arrangement, as described above.

The above-cited patient seat and an above-cited magnetic resonance device are explained in more detail below. In other words, the patient seat and the magnetic resonance device explained in more detail below may e.g. be used with one or more of the above-explained RF shielding facilities in the above-explained configuration. Such a patient seat and such a magnetic resonance device are the subject matter of the European patent application 24159090.0. Reference is made in the present application to the details disclosed in said application in relation to the patent seat and/or to the magnetic resonance device, e.g. to the construction and the mechanical functionality of the patient seat and/or the magnetic resonance device, and incorporated by reference into the present disclosure.

### BRIEF DESCRIPTION OF THE DRAWINGS

All the embodiments described herein can be combined with one another unless explicitly stated otherwise. Further features, advantages and potential applications of the present disclosure will become apparent from the following description, the exemplary embodiment and the figures, in which:

FIG. 1 illustrates an embodiment of a magnetic resonance device comprising an exemplary shielding facility, in accordance with one or more embodiments of the present disclosure;

FIGS. 2A and 2B illustrate embodiments of an exemplary magnetic resonance device, in accordance with one or more embodiments of the present disclosure;

FIG. 3 illustrates an embodiment of an exemplary patient seat, in accordance with one or more embodiments of the present disclosure;

FIG. 4 illustrates an embodiment of another exemplary patient seat, in accordance with one or more embodiments of the present disclosure; and

FIG. 5 illustrates another embodiment of a magnetic resonance device comprising an exemplary shielding facility, in accordance with one or more embodiments of the present disclosure.

### DETAILED DESCRIPTION OF THE DISCLOSURE

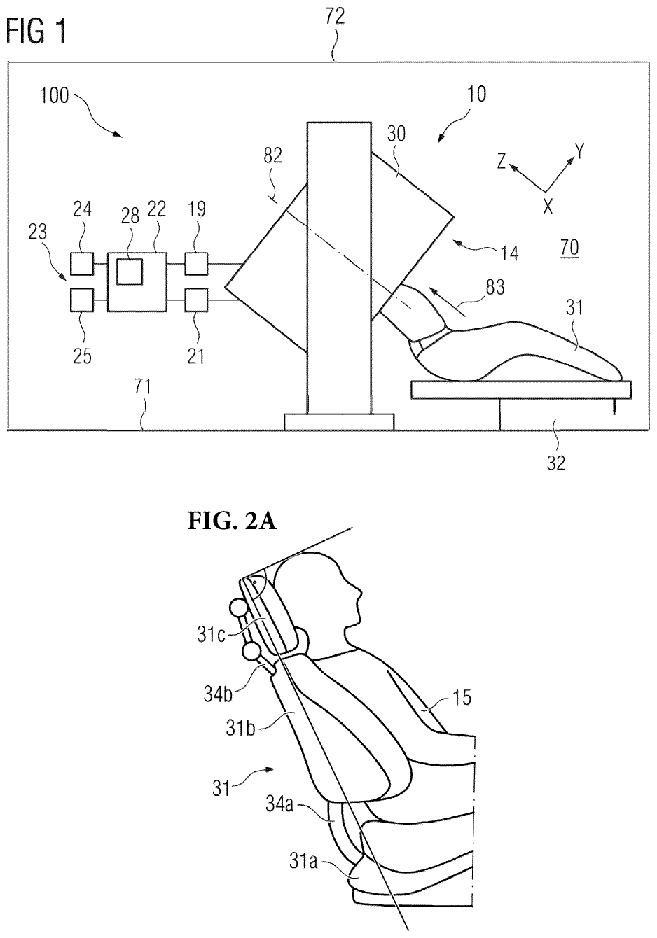

The embodiments of a patient seat and a magnetic resonance device described in FIGS. 1 to 5 may e.g. be used with a RF shielding facility for a dedicated magnetic resonance device, e.g. for a dedicated MRT head scanner or a dedicated extremities scanner.

FIG. 1 illustrates an embodiment of a magnetic resonance device comprising an exemplary shielding facility, in accordance with one or more embodiments of the present disclosure. Specifically, FIG. 1 shows an embodiment of a magnetic resonance device 10 of a magnetic resonance arrangement 100 according to the disclosure having a shielding facility in the form of a shielding cabin, which is provided by means of planar RF shielding elements 72. In this case, the RF shielding elements 72 may comprise for example a metallic fabric, for example a copper mesh, and/or metal plates, such as copper plates, for example, and/or other attenuating elements.

The magnetic resonance device 10 may for example be designed to conduct a magnetic resonance examination of any suitable patient region, e.g. a head region, a jaw region, and/or a dental region of a patient 15. As other examples, the magnetic resonance device 10 may equally be designed to perform cardiac imaging, neurological imaging, urological imaging, orthopedic imaging, prostate imaging, or an imaging examination of other body regions of the patient 15, such as extremities.

In the embodiment shown in FIG. 1, a longitudinal axis 82 of the patient receiving zone 14 (or a longitudinal axis 82 of the magnetic resonance device 10) is arranged inclined relative to a horizontal 71, e.g. a horizontally aligned floor surface 71 of an examination room 70 formed by a RF shielding element 72. In the present example, the patient access direction 83 coincides with the Z-direction of the magnetic resonance device 10 and is also inclined relative to the floor surface 71. The inclination of the magnetic resonance device 10 enables a magnetic resonance examination of a seated patient 15 and, consequently, permits a reduction in the space required by the magnetic resonance device 10 with the patient seat 31.

The requirements in respect of the shielding by means of the RF shielding elements 72 or, as the case may be, in respect of the room 70 constructed by said elements can be reduced precisely by means of a combination of a dedicated magnetic resonance device 10 with a patient seat 31 such that the RF shielding elements 72 can be designed more simply. The stability requirements in terms of an installation site can also be reduced as a result of the comparatively low weight of an illustrated dedicated magnetic resonance device 10 and an illustrated patient seat 31, such that a magnetic resonance arrangement 100 according to the disclosure can also be arranged for example in mezzanines in a building and not just, as is usual in practice, on specially reinforced floors.

In the present example, the patient seat 31 has a drive unit 32, which is designed to move the patient seat 31 variably along a spatial direction. In the example shown, the drive unit is designed to move the patient seat 31 parallel to the floor surface 71. It is, however, also conceivable that the drive unit is designed to move the patient seat 31 at an angle to the floor surface 71, e.g. along the Z-direction of the magnetic resonance device 10. The drive unit can also be designed to move the patient seat 31 variably along several spatial directions, e.g. spatial directions oriented orthogonally to one another.

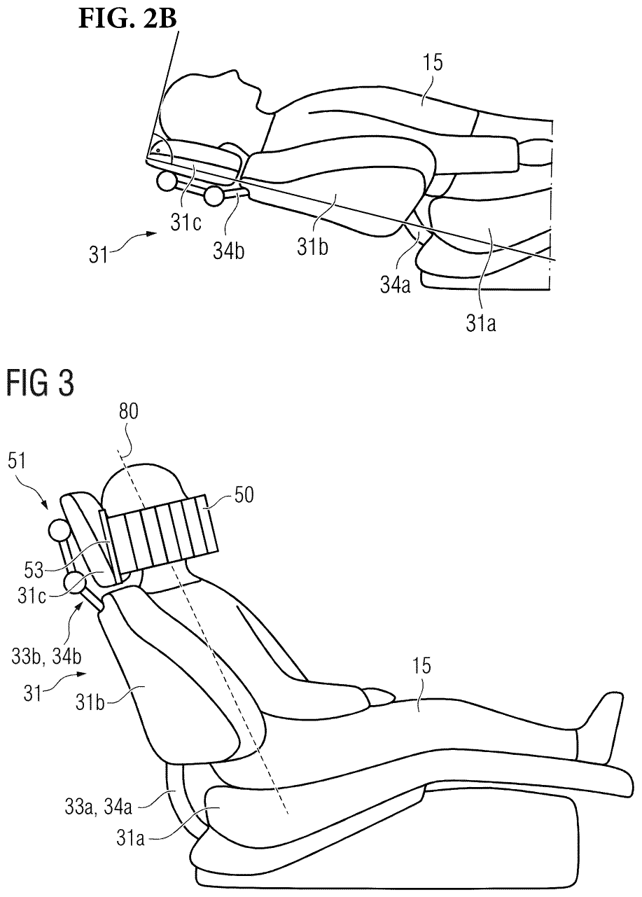

FIGS. 2A and 2B illustrate embodiments of an exemplary magnetic resonance device, in accordance with one or more embodiments of the present disclosure. Specifically, FIG. 2A and FIG. 2B show an embodiment of the patient seat 31 with a seat surface 31a, a backrest 31b, and a headrest 31c. The seat surface 31a and the backrest 31b are connected by means of a connecting element 34a, while the backrest 31b and the headrest 31c are connected by means of a connecting element 34b. A patient 15 is positioned on the patient seat 31 in a manner appropriate for the application.

The connecting elements 34a and 34b may be designed to enable a variable relative movement between the sections 31a, 31b, and 31c of the patient seat 31. In the example shown, the connecting element 34a is embodied to enable a movement of the backrest 31b relative to the seat surface 31a. On the other hand, the connecting element 34b enables a movement of the headrest 31c relative to the backrest 31b. In the present case, the connecting element 34a comprises a joint which enables the backrest 31b to be tilted relative to the seat surface 31a along a predetermined movement trajectory. The connecting element 34b comprises a plurality of joints, which enable the headrest 31c to be positioned and tilted relative to the backrest 31b.

In the example shown in FIG. 2A, the patient 15 is substantially disposed in an upright sitting position. It is conceivable that the longitudinal axis 82 of the patient receiving zone 14 may be inclined such that a magnetic resonance examination of the patient 15 can be performed in the upright sitting position. The patient seat 31 may e.g. be designed to avoid an arrangement of the head of the patient 15 relative to the headrest 31c when the patient 15 is leaning back on the patient seat 31 as shown in FIG. 2b. This enables steps in the preparation of the patient 15, such as an arrangement of local coils 26 or antenna elements 50, for example, to be carried out already on a patient 15 sitting upright, as a result of which the workload for medical staff can be reduced.

In an embodiment, the connecting elements 34a and 34b are designed to move the headrest 31c and the backrest 31b variably relative to the seat surface 31a, such that a position of the head of the patient 15 relative to the headrest 31c remains substantially unchanged when the patient 15 is leaning back. As shown in FIG. 2a and FIG. 2b, an angle between the headrest 31c and a tangent to the highest point of the head of the patient 15 remains substantially unchanged when the patient 15 leans back on the patient seat 31.

In all of the embodiments described herein, the patient seat 31 according to the disclosure may comprise passive or purely mechanical connecting elements 34, which enable a relative movement between the seat surface sections 31a, 31b and/or 31c (31a-c) by interaction of a patient 15 or a member of the medical staff. Similarly, the connecting elements 34 can be coupled to active positioning units 33, which have a drive and permit an automated relative movement of the sections 31a, 31b and/or 31c.

FIG. 3 illustrates an embodiment of an exemplary patient seat, in accordance with one or more embodiments of the present disclosure. Specifically, FIG. 3 shows an embodiment of a patient seat 31 having a guide element 53, which is designed to move the antenna element 50 of the radiofrequency unit 51 variably relative to the headrest 31c.

In this example, the guide element 53 and the antenna element 50 are mechanically connected to the headrest 31c of the patient seat 31. The guide element 53 is implemented as a hinge which enables the antenna element 50 to pivot or rotate about an axis defined by the guide element 53 (e.g. an axis aligned parallel to a sagittal plane and/or a frontal plane of the upper body of the patient 15). Thus, the antenna element 50 can be placed on the head of the patient 15 from the side or be positioned at a predetermined distance from the head of the patient 15.

In an embodiment, the patient seat 31 has at least two antenna elements 50 (not shown), which are arranged on opposite sides of the headrest 31c and flank the head of the patient 15 positioned on the patient seat 31 appropriately for the application from opposite sides. It is conceivable that the guide element 53 and/or the headrest 31c have/has a bearing, which is designed to move the antenna element 50 relatively along a parallel to the sagittal plane and/or the frontal plane of the upper body of the patient 15, e.g. along a parallel to an intersection line 80 of the sagittal plane with the frontal plane. Alternatively or in addition, it is conceivable that a guide element 53 is arranged with an antenna element 50 on the backrest 31b and/or the seat surface 31a to enable a magnetic resonance examination of further or other body regions of the patient 15.

In the embodiment of the patient seat 31 shown in FIG. 3, the headrest 31c, the backrest 31b and the seat surface 31a can be moved variably relative to one another by a patient 15 or a member of the medical staff by means of the connecting elements 34a and 34b. It is, however, likewise conceivable that one or more connecting elements 34 are designed as positioning units 33 or comprise a positioning unit 33. As a result, a variable relative movement of the headrest 31c, the backrest 31b, and/or the seat surface 31a can be performed in an automated manner. The positioning units 33a and/or 33b shown in FIG. 3 can, for example, be actuated or activated by means of drives integrated in the patient seat 31 as the result of an actuation by the control unit 22.

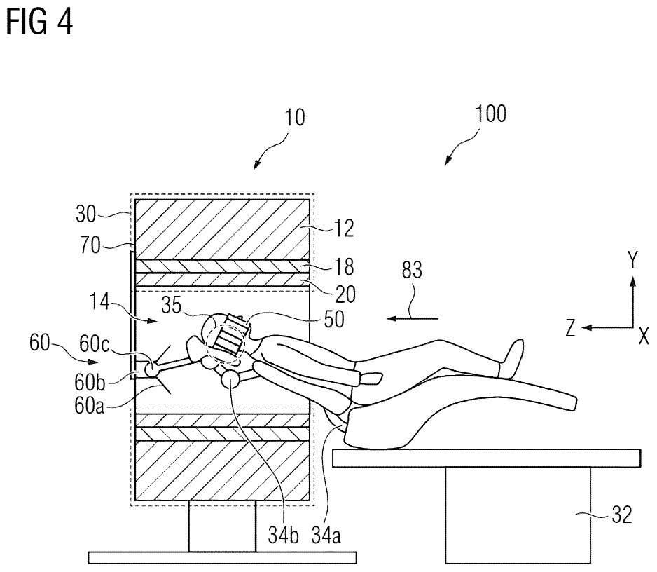

FIG. 4 illustrates an embodiment of another exemplary patient seat, in accordance with one or more embodiments of the present disclosure. Specifically, FIG. 4 shows an embodiment of a magnetic resonance device 10 comprising a locking mechanism 60. The locking mechanism 60 has a funnel or cone 60a with a cylindrical recess 60b and a coupling element 60c with a ball-shaped end. The cone 60a and the coupling element 60c are designed as complementary to one another and are embodied to mechanically engage with one another. In the present example, the coupling element 60c is mechanically connected to the patient seat 31, e.g. to the headrest or the connecting element 34b. The cone 60a is arranged inside the patient receiving zone 14 and mechanically connected to a magnet holding structure 70 of the magnetic resonance device 10.

The locking mechanism 60 is designed to limit a movement of a section of the patient seat 31 along a spatial direction. For example, the locking mechanism 60 prevents a movement of the patient seat 31 in the direction of the end of the patient receiving zone 14 with the locking mechanism 60 when the coupling element 60c abuts the wall in the cylindrical recess 60b. Furthermore, a freedom of movement of the ball-shaped end of the coupling element 60c along the Y-direction can be limited by the lateral surface of the cylindrical recess 60b. However, the coupling element 60b can be attached to a section of the patient seat 31 with a joint such that a restricted positioning of the headrest and/or the backrest of the patient seat along the Y-direction is enabled by means of the connecting elements 34b and/or 34a. Furthermore, the ball-shaped end of the coupling element 60c can be mounted rotatably in the cylindrical recess 60b in order to enable a restricted positioning of the headrest and/or the backrest of the patient seat along the Y-direction. Herein, the rotation of the ball-shaped end of the coupling element 60c can be limited by an opening angle of the cone 60a. In the embodiment shown in FIG. 4, the locking mechanism 60 is designed as passive. An engagement of the coupling element 60c in the cone 60a and/or the cylindrical recess 60b is therefore substantially realized by a movement of the patient seat 31 along the Z-direction by means of the drive unit 32 and/or a positioning unit 33.

It is, however, likewise conceivable that the cone 60a is coupled to the cylindrical recess 60b and/or the coupling element 60c is coupled to a drive which is embodied to move the two parts of the locking mechanism 60 toward one another. For example, the coupling element 60c can have a spindle-shaped shaft which enables the ball-shaped end to be positioned along the Z-direction by means of a gearing mechanism. In a further example, the patient seat 31 can have a hydraulic or pneumatic drive, e.g. a piston. Such a drive can be designed to deflect the coupling element 60c along the Z-direction. It is, however, likewise conceivable that the cone 60a can be positioned along the Z-direction and/or the Y-direction by means of a suitable drive.

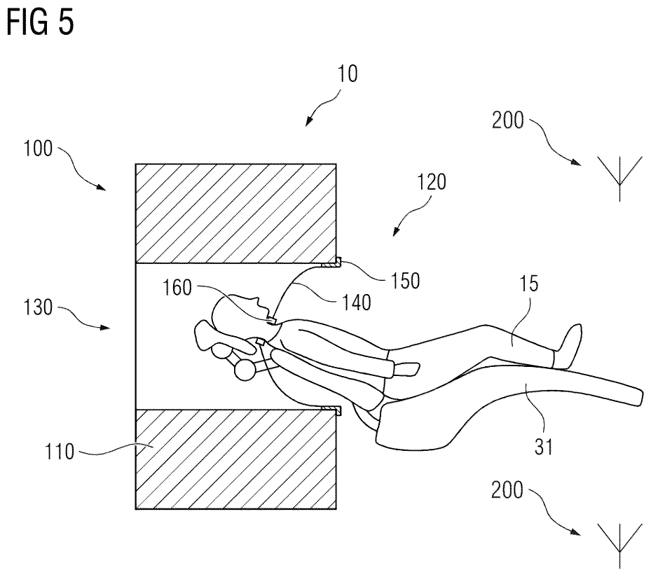

FIG. 5 illustrates another embodiment of a magnetic resonance device comprising an exemplary shielding facility, in accordance with one or more embodiments of the present disclosure. Specifically, FIG. 5 shows an embodiment of a magnetic resonance arrangement 100 comprising a magnetic resonance device 10 having a magnet 110 and a scanning bore opening 120 in which a patient 15 is partly arranged on an above-described patient seat 31 or has been introduced into the magnet 110. As can be clearly seen in FIG. 5, the side 130 of the magnet 110 opposite the scanning bore opening 120 is closed, the closed side 130 already being adequately shielded.

As shown in FIG. 5, a RF shield 140, in this case in the form of a copper mesh or fabric 140, is provided on the patient 15 and at the scanning bore opening 120 to shield off the scanning bore opening 120 with a patient 15 arranged therein. The RF shield 140 can be arranged for example at the scanning bore opening 120 via a first fastening means 150 and on the patient 15 via a second fastening means 160. The first fastening means 150 can be secured for example circumferentially on the scanning bore opening 120, and the second fastening means 160 can be arranged for example around the neck of the patient 15.

During the positioning, the RF shield 140, embodied for example as a stopper-like ring structure, can be moved together with the patient seat 31 in the magnetic resonance device 10. The RF shield 140 can for example be secured to a metal mesh which is advantageously covered with soft material or embedded therein and which either completely covers the body, the arms and the legs of the patient, or can also be wrapped around the neck of the patient 15 during the positioning. The RF shield 140 and the patient 15 may advantageously be grounded to further suppress a RF coupling.

It is furthermore possible to provide a shielding cabin 70 (shown in FIG. 1) in addition around the arrangement shown in FIG. 5, which shielding cabin 70 can be provided by means of RF shielding elements 72. In addition or alternatively, it is also possible to provide RF antennas 200, which neutralize (e.g. via at least partial cancellation) the emitted radiation by means of destructive interference. In the illustrated embodiment of the dedicated magnetic resonance device 10 having a one-sided scanning bore opening 120, there is basically only the need to shield the side with the scanning bore opening 120 against radiofrequency interference. As a result, it is possible to arrange the room to be constructed by means of the modular RF shielding elements 72 such that it does not contain the magnetic resonance device 10 completely but covers only the part of the magnetic resonance device 10 having the scanning bore opening 120. This affords the opportunity to achieve a further reduction in the necessary installation space for a shielding cabin.

The present disclosure is not limited to the embodiment described in the foregoing as long as it is encompassed by the subject matter of the following claims. It is pointed out in addition that the terms “comprising” and “having” do not rule out other elements or steps and the indefinite articles “a” or “an” do not rule out a plurality. It is furthermore pointed out that features or steps which have been described with reference to the above embodiments may also be used in combination with other features.

It is furthermore pointed out that independent of the grammatical term usage, individuals with male, female, or other gender identities are included within the term.

Additionally, the various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently or configured to execute instructions or computer programs that are stored on a suitable computer-readable medium. Regardless of the particular implementation, such units, etc., as applicable and relevant, may alternatively be referred to herein as “circuitry,” “controllers,” “processors,” or “processing circuitry,” or alternatively as noted herein.

What is claimed is:

1. A radio frequency (RF) shielding facility for a magnetic resonance tomography (MRT) head scanner, comprising:
a plurality of modular RF shielding elements configured to be connected to one another in an interconnected state to form a space for arranging the MRT head scanner;
an RF shield having a first side configured to be arranged on a patient and/or on a movable patient seat and a second side configured to be arranged on the MRT head scanner; and
an RF antenna configured to at least partially cancel an RF signal of the MRT head scanner via destructive interference.

2. The RF shielding facility as claimed in claim 1, wherein the plurality of modular RF shielding elements comprise different planar components and are configured as (i) door elements, (ii) window elements, and (iii) wall, ceiling, and floor elements.

3. A magnetic resonance imaging system, comprising:
a magnetic resonance tomography (MRT) imaging device; and
a radio frequency (RF) shielding facility for an MRT head scanner of the MRT imaging device, the RF shielding facility comprising:
a plurality of modular RF shielding elements configured to be connected to one another in an interconnected state to form a space for arranging the MRT head scanner;
an RF shield having a first side configured to be arranged on a patient and/or on a movable patient seat, and a second side configured to be arranged on the MRT head scanner; and
an RF antenna configured to at least partially cancel an RF signal of the MRT head scanner via destructive interference.

4. The magnetic resonance imaging system as claimed in claim 3, wherein the MRT imaging device comprises a one-sided scanning bore opening configured to accommodate at least a patient's head.

5. The magnetic resonance imaging system as claimed in claim 4, wherein a side of the MRT imaging device opposite the scanning bore opening is fixedly closed and shielded against RF interference.

6. The magnetic resonance imaging system as claimed in claim 4, wherein the RF shield is configured to provide RF sealing of the one-sided scanning bore opening.

7. The magnetic resonance imaging system as claimed in claim 3, wherein the plurality of modular RF shielding elements are connected to one another in the interconnected state to form the space around the MRT imaging device and the movable patient seat.

8. The magnetic resonance imaging system as claimed in claim 4, wherein the plurality of modular RF shielding elements are connected to form the space around the one-sided scanning bore opening of the MRT imaging device and the movable patient seat, and
wherein a side of the MRT imaging device opposite the scanning bore opening is not within the space.

9. The magnetic resonance imaging system as claimed in claim 3, wherein the plurality of modular RF shielding elements form a shielding cabin.
